(19) United States
(12) Patent Application Publication
(10) Pub. No.: US 2024/0316302 A1
(43) Pub. Date: Sep. 26, 2024

(54) METHOD AND SYSTEM FOR EXCHANGING A TRACHEOSTOMY

(71) Applicant: Mayo Foundation for Medical Education and Research, Rochester, MN (US)

(72) Inventors: David Abia Trujillo, St. Johns, FL (US); Sebastian Fernandez-Bussy, Ponte Vedra Beach, FL (US)

(73) Assignee: Mayo Foundation for Medical Education and Research, Rochester, MN (US)

(21) Appl. No.: 18/605,093

(22) Filed: Mar. 14, 2024

Related U.S. Application Data
(60) Provisional application No. 63/454,425, filed on Mar. 24, 2023.

(51) Int. Cl. A61M 16/04; A61M 16/00; A61M 39/10

(52) U.S. Cl. CPC A61M 16/0465; A61M 16/0003; A61M 39/10; A61M 2205/073; A61M 2209/00; A61M 2210/065

(57) ABSTRACT

The present disclosure relates to a tracheostomy exchange system including a catheter comprising a distal end and a proximal end, a balloon fluidly coupled to the distal end of the catheter, and an adapter assembly removably coupleable to the catheter and configured to fluidly couple the balloon with the proximal end of the catheter when the adapter assembly is coupled to the catheter. When the adapter assembly is decoupled from the catheter, the balloon is fluidly sealed from the proximal end of the catheter. When the is inflated before the adapter assembly is decoupled from the catheter, the balloon can remain inflated while the tracheostomy is exchanged preventing aerosolization, protecting the lungs from bleeding and aspiration, allowing for effective oxygenation while preserving an access to the main airway.

### CROSS-REFERENCE TO RELATED APPLICATION

The priority benefit of U.S. Patent Application Ser. No. 63/454,425, filed on Mar. 24, 2023, is hereby claimed and the entire contents are incorporated by reference.

### TECHNICAL FIELD

The present disclosure relates to a tracheostomy, and more particularly, to a tracheostomy exchange system.

### BACKGROUND

Patients with acute and chronic respiratory failure require a tracheostomy tube for breathing, and must undergo a tracheostomy replacement every two to three months. During these replacements (also known as tracheostomy exchanges), patients encounter risks that compromise their safety and recovery. Currently, a tracheostomy patient must be transferred from a long-time care facility or their home to a hospital for a replacement procedure. Many risks accompany a tracheostomy replacement procedure, such as airway loss, massive bleeding, inability to properly ventilate, aspiration, or false airway lumen creation. For example, when a tracheostomy is removed and a flexible catheter is inserted to maintain an airway, the catheter is not rigid enough to maintain or seal the air pathway or prevent aspiration. Additionally, a tracheostomy replacement is considered an aerosol-generating procedure and there is no system that prevents aerosolization while exchanging a tracheostomy. With the dangers of COVID-19 and other air-transmissible viruses, aerosolization presents a hazardous exposure for the medical staff exchanging a tracheostomy.

### SUMMARY

The present disclosure relates to a system and method for safely replacing a tracheostomy.

In a first example aspect, a tracheostomy exchange system, the system comprising a catheter comprising a distal end and a proximal end. A balloon may be fluidly coupled to the distal end of the catheter. An adapter assembly may be removably coupleable to the catheter and configured to fluidly couple the balloon with the proximal end of the catheter when the adapter assembly is coupled to the catheter. When the adapter assembly is decoupled from the catheter, the balloon may be fluidly sealed from the proximal end of the catheter such that when the balloon is inflated, the balloon remains inflated after the adapter assembly is decoupled.

In a second example aspect, a method of replacing a tracheostomy may include inserting a tracheostomy exchange system through a hollow center of a tracheostomy implanted into a subject. The tracheostomy exchange system may include a catheter having a distal end, a proximal end, and a hollow cavity. An inflatable balloon may be coupled to the distal end of the catheter. The method may include inflating the balloon disposed at the distal end of the catheter, and removing the tracheostomy from the subject while the balloon remains inflated.

In accordance with any one of the first and second example aspects, the tracheostomy exchange system and method of replacing a tracheostomy may include any one or more of the following forms.

In one example, a seal may be coupled to the catheter and may be movable between a collapsed configuration and an expanded configuration.

In another example, the catheter may include a longitudinal axis, a first lumen aligned with the longitudinal axis, and a second lumen radially offset from the longitudinal axis.

In some examples, the balloon may be in fluid communication with the second lumen.

In other forms, the balloon may be inflatable by pumping air into the second lumen using an external inflation device.

In yet another form, when the seal is in the expanded configuration, the seal may cover an opening in fluid communication with the second lumen of the catheter.

In some forms, when the seal is in the collapsed configuration, the seal may expose the port in fluid communication with the second lumen of the catheter.

In some aspects, the proximal end of the catheter may include a coupler removably coupleable to the seal.

In other aspects, the adapter assembly may include an adapter body including a cavity.

In some examples, an adapter seal may be disposed within the cavity of the adapter body.

In on example, the coupler of the catheter may be removably coupleable to the adapter seal of the adapter assembly.

In another aspect, the coupler may include an indentation sized to receive an annular ring of the adapter seal of the adapter assembly and an annular ring of the seal.

In some forms, when the balloon is inflated, the balloon may have a diameter in a range of approximately 25 mm to approximately 35 mm.

In other forms, the balloon may have a length in a range of approximately 15 mm to approximately 25 mm.

In another form, the method may include inserting a second tracheostomy over the proximal end of the catheter into the subject.

In yet another form, the method may include deflating the balloon after the inserting the second tracheostomy.

In some examples, the method may include removing the tracheostomy exchange system while the second tracheostomy remains in place.

In one aspect, the method may include inflating an inflatable device coupled to the second tracheostomy.

In another aspect, the method may include deflating a balloon of the tracheostomy before removing the tracheostomy.

In other aspects, the method may include removing a proximal end adapter of the tracheostomy exchange system.

In some forms, the proximal end of the catheter may be sealed when engaged with the proximal end adapter and sealed when disengaged from the proximal end adapter.

In another form, the method may include sealing the proximal end of the catheter with an expandable seal when the catheter disengages from the proximal end adapter.

Other features and advantages of the present disclosure will be apparent from the following detailed description, the figures, and the claims.

### BRIEF DESCRIPTION OF THE DRAWINGS

FIG. 1 is a side view of a tracheostomy exchange system assembled in accordance with the teachings of the present disclosure, showing the exchange system fully assembled;

FIG. 2 is a side view of the tracheostomy exchange system of FIG. 1, showing the system partially disassembled;

FIG. 3 is a side, cross-sectional view of a proximal end of the system of FIG. 1;

FIG. 4 is a magnified view of the proximal end of the system taken at section D of FIG. 3;

FIG. 5 is a side, cross-sectional view of the proximal end of the system of FIG. 2;

FIG. 6 is a magnified view of the proximal end of the system taken at section C of FIG. 5;

FIG. 7 is a perspective view of a proximal end adapter of the system of FIG. 1;

FIG. 8 is a side view of the adapter of FIG. 4;

FIG. 9 is a cross-sectional view of the adapter of FIG. 8;

FIG. 10 is a side view of a catheter seal of the system of FIG. 1;

FIG. 11 is a cross-sectional view of the catheter seal of FIG. 10;

FIG. 12 is a side view of a catheter of the system of FIG. 1;

FIG. 13 is a cross-sectional view of the catheter of FIG. 12;

FIG. 14 is a flow chart of a method of replacing a tracheostomy using a tracheostomy exchange system in accordance with the teachings of the present disclosure;

FIG. 15 is a partial image of a tracheostomy and a tracheostomy exchange system inside a subject and assembled in accordance with the teachings of the present disclosure;

FIG. 16 is the tracheostomy and tracheostomy exchange system of FIG. 15, showing an inflated distal balloon;

FIG. 17 is the tracheostomy and tracheostomy exchange system of FIG. 15, showing the adapter removed from a proximal end of the catheter; and

FIG. 18 is the tracheostomy exchange system of FIG. 17, showing the tracheostomy removed from the subject.

### DETAILED DESCRIPTION

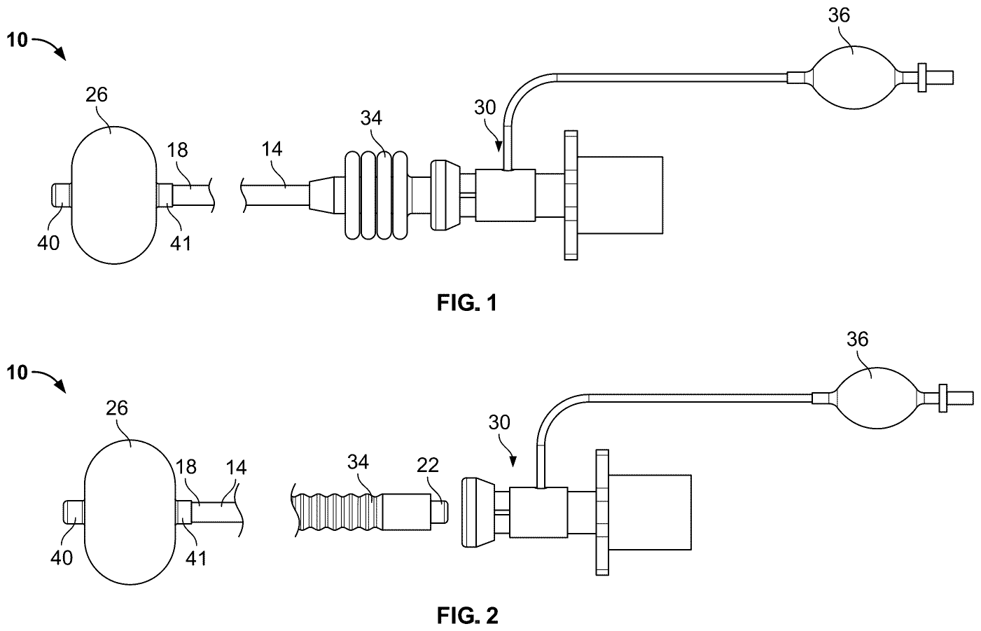

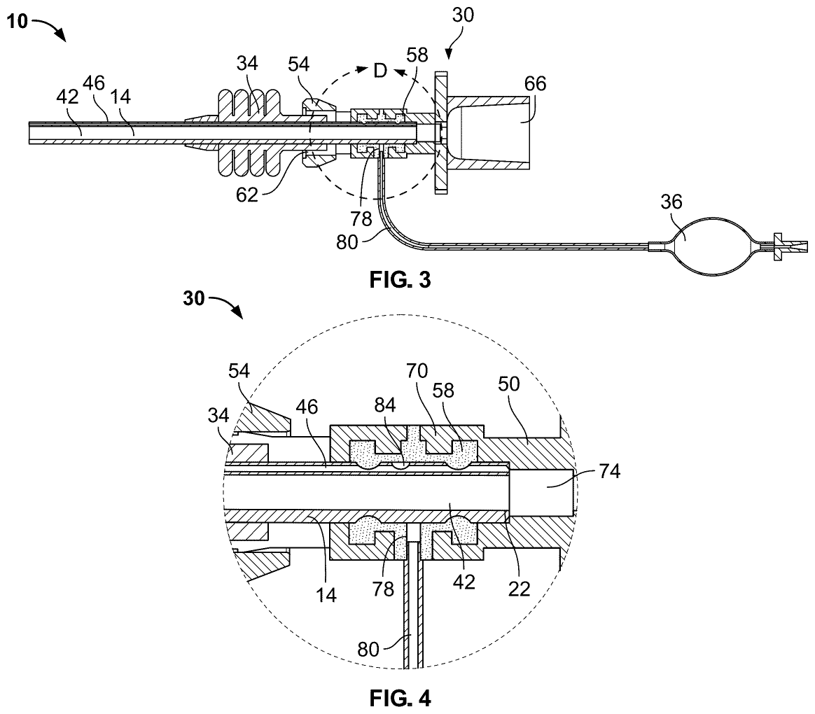

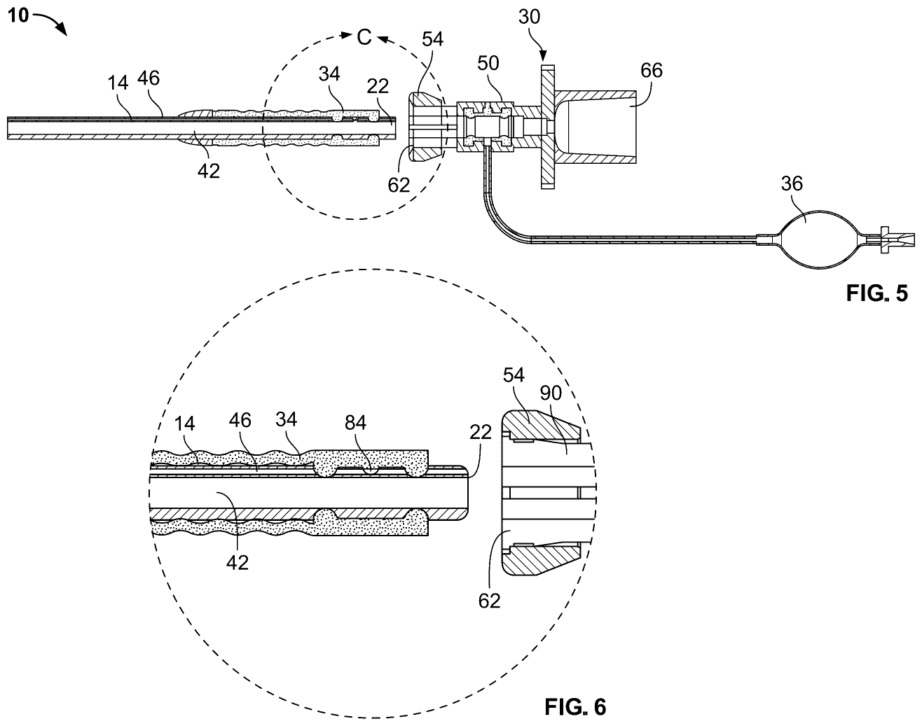

The present disclosure relates to a system and method for safely replacing a tracheostomy. In FIGS. 1 and 2, an example tracheostomy exchange system 10 includes a catheter 14 comprising a distal end 18 and a proximal end 22, a balloon 26 fluidly coupled to the distal end 18 of the catheter 14, and an adapter assembly 30 removably coupleable to the proximal end 22 of the catheter 14. The system also includes a seal 34 coupled to the catheter 14 and that is movable between a collapsed configuration, as shown in FIGS. 1 and 3, and an expanded configuration, as shown in FIGS. 2, 5, and 6. An inflation device 36 is coupled to the adapter assembly 30 for inflating the balloon 26. The inflation device 36 has a catheter lumen with a balloon that indicates to the user that the balloon 26 is indeed inflated or deflated. The distal end of the inflation device 36 connects to a syringe to allow user to inflate the distal balloon 26. The balloon 26 and the catheter 14 are configured for insertion into a subject (e.g., a human patient), and the seal 34, adapter assembly 30, and inflation device 36 remain outside of the subject during a tracheostomy exchange. Turning briefly to FIG. 3, the catheter 14 includes a first lumen 42 that provides an airway for the subject, and a second lumen 46 offset from the first lumen that fluidly connects the catheter 14 to the balloon 26 and to the inflation device 36 when the adapter assembly 30 is securely connected to the catheter 14.

When the balloon 26 is inflated, the balloon 26 has a diameter D in a range of approximately 25 mm to approximately 35 mm, and a length in a range of approximately 15 mm to approximately 25 mm. The dimensions of the balloon 26 may be reduced for the anatomy of a younger or smaller subject. On either side of the inflated balloon, the system 10 includes first and second radiopaque markers 40, 41 for visibility under fluoroscopy.

In FIGS. 1, 3, and 4, the adapter assembly 30 is configured to fluidly couple the balloon 26 with the proximal end 22 of the catheter 14 when the adapter assembly 30 is coupled to the catheter 14. Referring to FIG. 4, a tube of the inflation device is in fluid communication with the balloon 26 by way of the second lumen 46 of the catheter 14. In FIGS. 2, 5, and 6, when the adapter assembly 30 is decoupled from the catheter 14, the balloon 26 is fluidly sealed from the proximal end 22 of the catheter 14. Referring to FIG. 6, the seal 34 is in sealing engagement with the second lumen 46 of the catheter so that the balloon 26 can remain inflated after the adapter assembly 30 is removed from the proximal end 22 of the catheter 14.

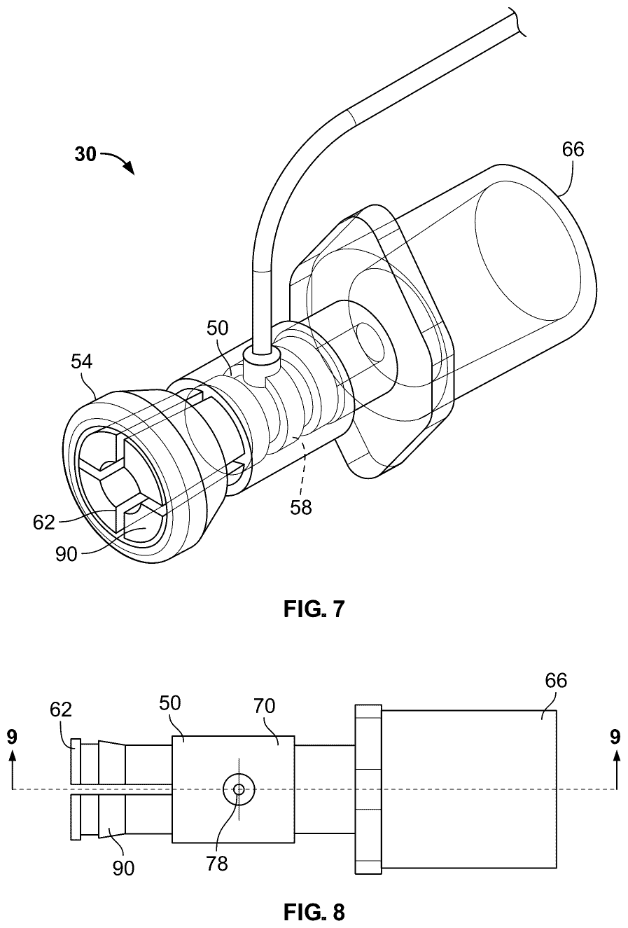

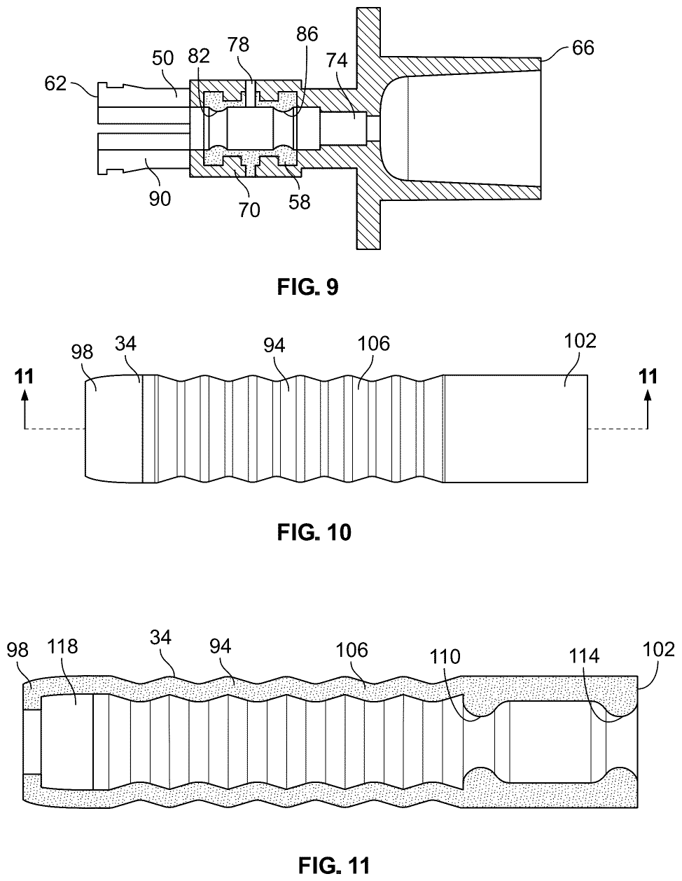

Referring now to FIGS. 7-9, the adapter assembly 30 includes a body 50, a collar 54, and an adapter seal 58. The body 50 has a distal end 62, a proximal end 66, an intermediate portion 70 disposed between the distal and proximal ends 62, 66, and a cavity 74 extending between the distal and proximal ends 62, 66. The proximal end 66 of the adapter body 50 defines a fitting that is sized and shaped to attach to a corresponding fitting of an Ambu® bag or an external air supply. The cavity 74 is fluidly coupled with the first lumen 42 of the catheter 14 when the adapter assembly 30 is coupled to the catheter 14.

Additionally, the adapter assembly 30 is configured to fluidly couple the inflation device 36 with the balloon 26. Specifically, the body 50 includes a port 78 at the intermediate portion 70. The port 78 is arranged to fluidly couple with a tube 80 of the inflation device 36 with the second lumen 46 of the catheter 14. Turning briefly to FIG. 4, an opening 84 disposed at the proximal end 22 of the catheter 14 fluidly couples the port 78 of the adapter assembly 30 with the second lumen 46.

The adapter seal 58 is disposed inside the cavity 74 of the body 50 and is adjacent to the intermediate portion 70. The adapter seal 58 includes a first annular ring 82 and a second annular ring 86 that are respectively disposed distally and proximally relative to the port 78. The annular rings 82, 86 slightly extend into the cavity 74 of the body 50 and are configured to engage with a coupler of the catheter 14 to seal the catheter 14 when engaged (FIG. 4). The distal end 62 includes a plurality of flexible arms 90 (e.g., four arms) that are deformable when engaged with the collar 54. The collar 54 is disposed at the distal end 62 of the body 50 and is configured to sealably secure the distal end 62 to the catheter 14 by biasing the flexible arms 90 inwardly against the catheter 14 and/or the seal 34 by press-fit.

In FIGS. 10 and 11, the seal 34 includes a body 94 including a distal end 98, a proximal end 102, and a collapsible portion 106. The body 94 is sized to fit around an exterior surface of the catheter 14 at the proximal end 22 of the catheter. The distal end 98 of the seal 34 does not move relative to the catheter 14, and the proximal end 102 moves relative to the catheter 14 between the expanded and collapsed configurations. At the proximal end 102, the seal 34 includes a first annular ring 110 and a second annular ring 114 that slightly extend into a cavity 118 of the body 94. Similar to the first and second annular rings 82, 86 of the adapter seal 58, the annular rings 110, 114 of the seal 34 are configured to engage with the coupler of the catheter 14 to seal the second lumen 46 of the catheter 14. Turning briefly to FIG. 6, when the seal 34 is in the expanded configuration, the seal 34 replaces the adapter seal 58, covers the opening 84 of the catheter 14, and engages the annular rings 110, 114 with the coupler. The collapsible portion 106 is configured to expand from the collapsed position immediately after the adapter assembly 30 decouples from the proximal end 22 of the catheter 14.

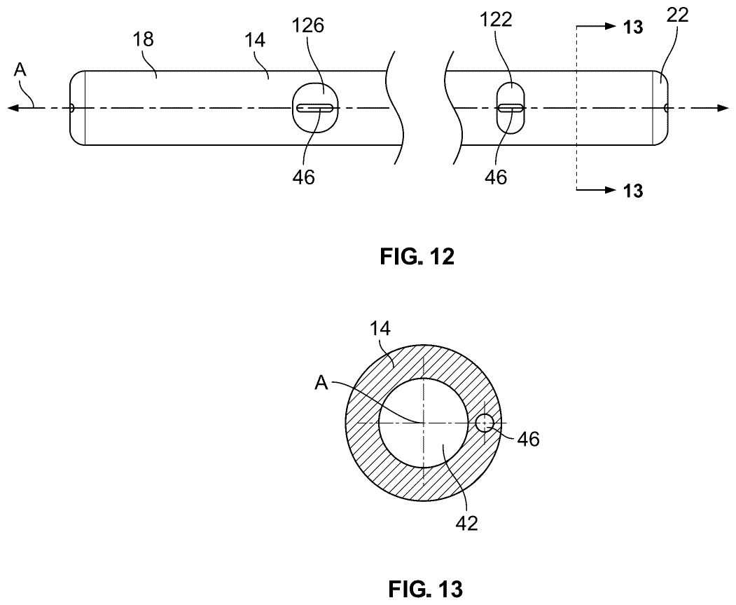

Turning now to FIGS. 12 and 13, the catheter 14 includes a longitudinal axis A, the first lumen 42 aligned with the longitudinal axis A, and the second lumen 46 radially offset from the longitudinal axis A. The proximal end 22 of the catheter 14 includes a coupler that is removably coupleable to the seal 34 and adapter seal 58. The coupler includes an indentation 122 that is sized to receive one of the annular rings 82, 86 of the adapter seal 58 of the adapter assembly 30 and one of the annular rings 110, 114 of the seal 34. At the distal end 18 of the catheter, an opening 126 in the catheter 14 is arranged for positioning inside a cavity of the balloon 26 to fluidly coupled the second lumen 46 with a bladder of the balloon 26.

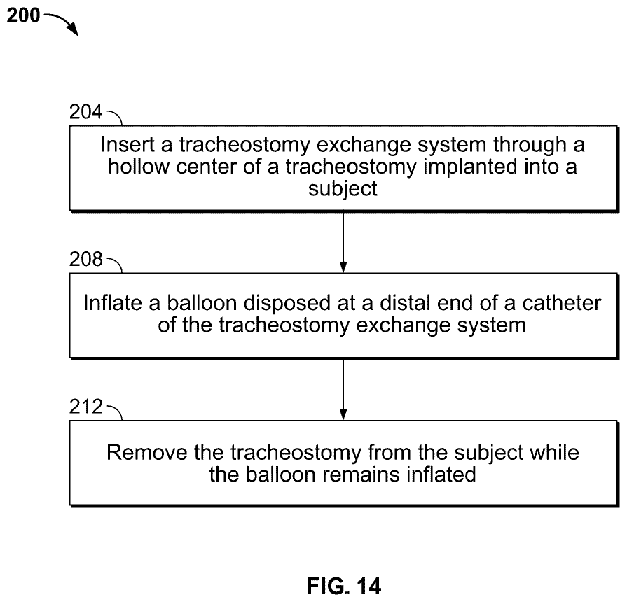

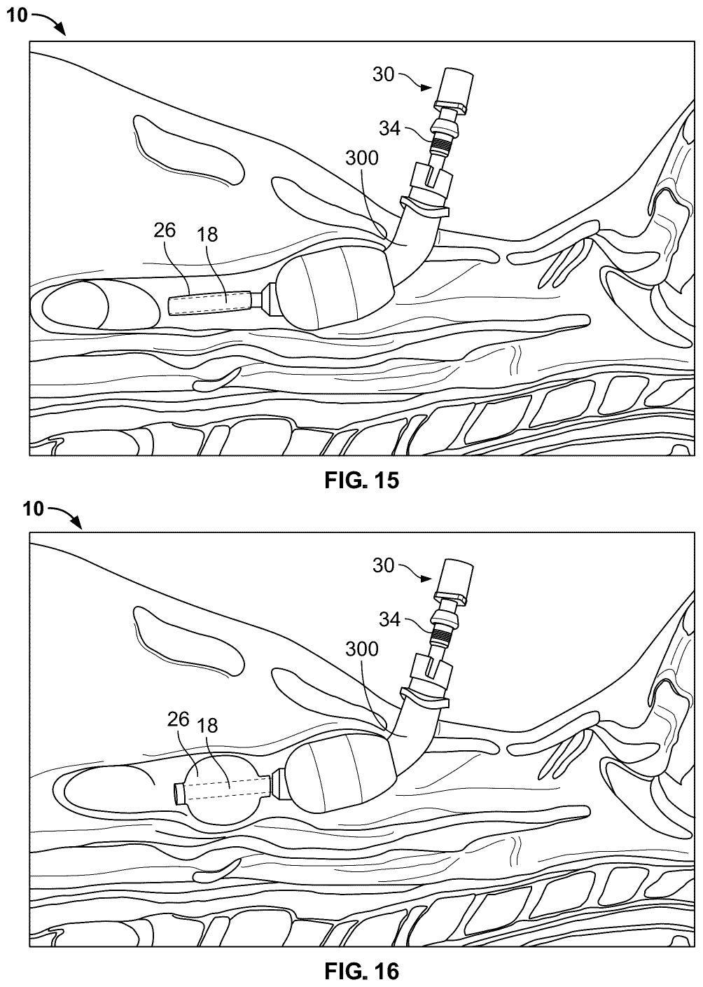

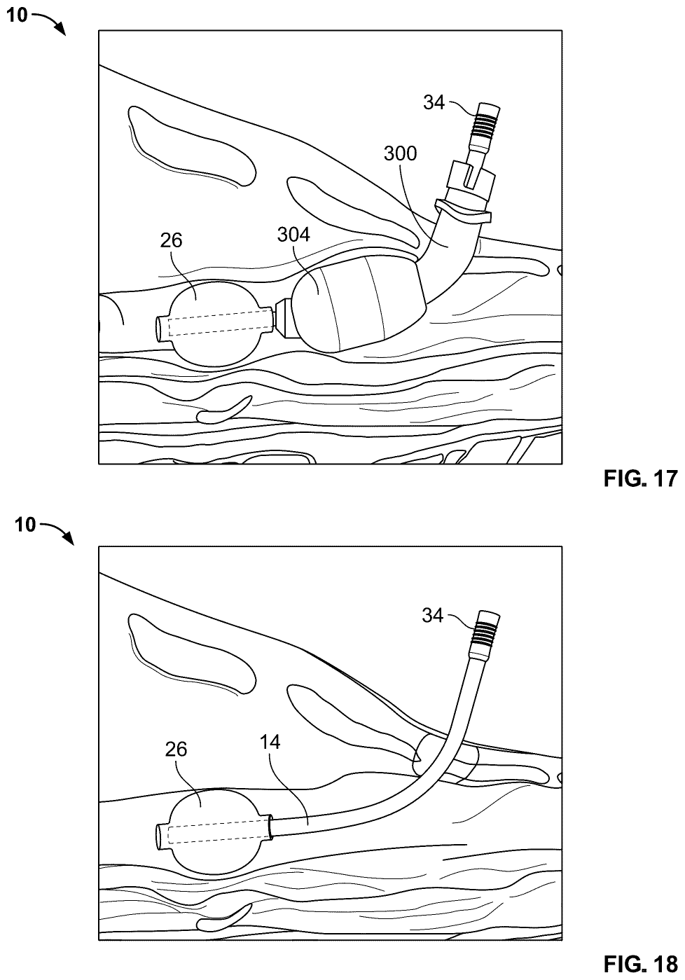

In FIG. 14, a flow chart represents an example method 200 for exchanging a tracheostomy using the tracheostomy system 10 of FIGS. 1-13, and will be described with reference to FIGS. 15-18. The method 200 includes a first step 204 of inserting a tracheostomy exchange system 10 through a hollow center of a tracheostomy 300 implanted into a subject, as shown in FIG. 15. The tracheostomy exchange system 10 includes a catheter 14 having a distal end 18, a proximal end 22, and an inflatable balloon 26 coupled to the distal end 18 of the catheter 14. A second step 208 includes inflating the balloon 26 disposed at the distal end 18 of the catheter 14, as shown in FIG. 16. Subsequently, the adapter assembly 30 is removed from the catheter 14. When the balloon 26 is sufficiently inflated, the method 200 includes a step 212 of removing the tracheostomy 300 from the subject while the balloon 26 remains inflated, as shown in FIG. 17.

To inflate the balloon 26, the method 200 may include a step of inflating the balloon of the inflation device 36 (hidden in FIGS. 15-18) that is coupled to the adapter assembly 30 using a syringe. After inflating the balloon 26, the method 200 may include decoupling the adapter assembly 30 from the catheter 14 as shown in FIG. 17. The seal 34 moves to an expanded configuration, as shown in FIGS. 17 and 18, to seal the catheter 14, thereby sealing the balloon 26 to keep the balloon 26 inflated. Before removing the existing tracheostomy 300, a balloon 304 of the existing tracheostomy 300 is deflated.

Additionally, the method 200 also includes inserting a second tracheostomy over the proximal end 22 of the catheter 14 and into the subject. After the second tracheostomy is disposed in the patient, the balloon 26 is deflated, and the tracheostomy exchange system can be removed while the second tracheostomy remains in place within the patient.

The system 10 and method 200 disclosed herein provide a number of advantages over current methods of exchanging a tracheostomy. A first advantage is maintaining an airway throughout the procedure. In case of massive bleeding, the system 10 can be used to tamponade the bleeding site or to isolate a bleeding source in the airway, avoiding blood spilling into the rest of the airway. The system allows proper sealing of the airway so that the tidal volume that is administered through the exchanger is efficiently ventilated and oxygenates the patient.

The balloon advantageously protects the airway from gastric or pharyngeal content spilling into the airway. Additionally, the balloon significantly reduces the aerosol emission on the subject's exhalation by remaining inflated without disrupting the airway. False airway lumen creation will not happen with the disclosed system 10 as the tracheostomy exchange system 10 works as a guidewire to the exchange of the tracheostomy.

While this specification contains many specific implementation details, these should not be construed as limitations on the scope of any disclosure or of what may be claimed, but rather as descriptions of features that may be specific to particular embodiments of particular disclosures. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. A tracheostomy exchange system, the system comprising:
a catheter comprising a distal end and a proximal end;
a balloon fluidly coupled to the distal end of the catheter;
an adapter assembly removably coupleable to the catheter and configured to fluidly couple the balloon with the proximal end of the catheter when the adapter assembly is coupled to the catheter;
wherein when the adapter assembly is decoupled from the catheter, the balloon is fluidly sealed from the proximal end of the catheter such that when the balloon is inflated, the balloon remains inflated after the adapter assembly is decoupled.

2. The system of claim 1, comprising a seal coupled to the catheter and movable between a collapsed configuration and an expanded configuration.

3. The system of claim 2, wherein the catheter comprises a longitudinal axis, a first lumen aligned with the longitudinal axis, and a second lumen radially offset from the longitudinal axis.

4. The system of claim 3, wherein the balloon is in fluid communication with the second lumen.

5. The system of claim 3, wherein the balloon is inflatable by pumping air into the second lumen using an external inflation device.

6. The system of claim 3, wherein when the seal is in the expanded configuration, the seal covers an opening in fluid communication with the second lumen of the catheter.

7. The system of claim 6, wherein when the seal is in the collapsed configuration, the seal exposes a port in fluid communication with the second lumen of the catheter.

8. The system of claim 2, wherein the proximal end of the catheter comprises a coupler removably coupleable to the seal.

9. The system of claim 8, wherein the adapter assembly comprises:
an adapter body including a cavity; and
an adapter seal disposed within the cavity of the adapter body;
wherein the coupler of the catheter is removably coupleable to the adapter seal of the adapter assembly.

10. The system of claim 9, wherein the coupler comprises an indentation sized to receive an annular ring of the adapter seal of the adapter assembly and an annular ring of the seal.

11. The system of claim 1, wherein when the balloon is inflated, the balloon has a diameter in a range of approximately 25 mm to approximately 35 mm.

12. The system of claim 1, wherein the balloon has a length in a range of approximately 15 mm to approximately 25 mm.

13. A method of replacing a tracheostomy, the method comprising:
inserting a tracheostomy exchange system through a hollow center of a tracheostomy implanted into a subject, the tracheostomy exchange system comprising a catheter having a distal end, a proximal end, and a hollow cavity; an inflatable balloon coupled to the distal end of the catheter;
inflating the balloon disposed at the distal end of the catheter;
removing the tracheostomy from the subject while the balloon remains inflated.

14. The method of claim 13, comprising inserting a second tracheostomy over the proximal end of the catheter into the subject.

15. The method of claim 14, comprising deflating the balloon after the inserting the second tracheostomy.

16. The method of claim 15, comprising removing the tracheostomy exchange system while the second tracheostomy remains in place.

17. The method of claim 15, comprising inflating an inflatable device coupled to the second tracheostomy.

18. The method of claim 13, comprising deflating a balloon of the tracheostomy before removing the tracheostomy.

19. The method of claim 13, comprising removing a proximal end adapter of the tracheostomy exchange system, wherein the proximal end of the catheter is sealed when engaged with the proximal end adapter and sealed when disengaged from the proximal end adapter.

20. The method of claim 19, comprising sealing the proximal end of the catheter with an expandable seal when the catheter disengages from the proximal end adapter.
